(19) United States
(12) Patent Application Publication
(10) Pub. No.: US 2002/0054851 A1
(43) Pub. Date: May 9, 2002

(54) 32P-Polyphosphazenes

(76) Inventors: Michael Grunze, Neckargemund (DE); Alexander Welle, Ladenburg (DE)

Correspondence Address: BURNS DOANE SWECKER & MATHIS L L P, POST OFFICE BOX 1404, ALEXANDRIA, VA, 22313-1404, US

(21) Appl. No.: 09/539,810

(22) Filed: Mar. 30, 2000

Related U.S. Application Data
(63) Continuation of application No. PCT/EP98/06167, filed on Sep. 29, 1998.

(30) Foreign Application Priority Data
Sep. 30, 1997 (DE) DE 197 43 373.1

(51) Int. Cl. A61M 36/14; A61K 51/00; A61M 36/00; C08G 79/02

(52) U.S. Cl. 424/1.81; 424/1.11; 424/1.25; 424/1.29; 424/1.37; 424/1.61; 424/1.65; 424/1.77; 600/1; 600/3; 600/7; 600/8; 528/168

(57) ABSTRACT

The invention concerns a radio-labeled antithrombogenic polymer and its use as part of therapeutic means to prevent excessive cell proliferation or scarring, and means which comprise the radiolabeled antithrombogenic polymer, such as emplastrum or artificial implants with a biocompatible coating.

### CROSS REFERENCE

This application is a continuation of International PCT Application PCT/EP98/06167 filed on Sep. 29, 1998, which designates the United States and is herein incorporated by reference in its entirety.

This invention concerns a radio-labeled antithrombogenic polymer and its use as a component of therapeutic means to prevent excessive cell proliferation or scarring, and means comprising the radio-labeled antithrombogenic polymer, such as emplastrum or artificial implants with a biocompatible coating.

One of the major complications from artificial implants is increased deposition of thrombocytes at the surface of the foreign body. Another is increased cell proliferation (scarring) of the injured and healing tissue involved with the artificial implant.

Production of thrombi when human blood comes into contact with a surface foreign to the body, such as artificial heart valves, is described at the state of the art (cf. informative material from the company Metronic Hall, Bad Homburg, Carmeda BioActive Surface (CBSA), pages 1-21, and Buddy D. Ratner, “The blood compatibility catastrophe”, Journal of Biomedical Materials Research, Vol. 27, 283-287; and Cary W. Akins, M. D., “Mechanical Cardiac Valvular Prostheses”, The Society of Thoracic Surgeons, 161-171, 1991). For instance, the commercial heart valves of pyrolyzed carbon now on the world market show increased tendency for thrombus development (cf. Cary W. Akins, above). At present not only do thromboses appear on implants which contact the blood, but there are also serious medical problems from emboli and inflammation (endocarditis).

With vascular implants, such as “stents”, there are not only the well-known problems of increased thrombus formation, but also restenoses (i. e., re-narrowing of the blood vessel in the region expanded by angioplasty, frequently the stent region). Those complications are initiated because of activation of the clotting and immune system by the implanted foreign object, and by damage to the vessel wall during implantation of the stent in the course of angioplasty. At present, therefore, patients with artificial heart valves are given clotting inhibitors (Vitamin K antagonists) as they are during postoperative treatment following angioplasty; but the dosages are problematical. It is impossible at this time to use stents in narrow (d<4 mm) and venous blood vessels because of thrombus formation. When they are implanted in arteries, strong tissue proliferation (intimal hyperplasia) often causes renewed constriction of vessels in the stent region (restenosis). The frequency of restenosis for the usual commercially available stents is about 30-50% within 6 months after successful angioplasty. Hehrlein et al. showed that the frequency of restenosis could be reduced significantly by use of radioisotope radiation. In their experiments, Hehrlein et al. used ³²P ions implanted by ion implantation in the metallic material of the stent (Ti/Ni alloys, tantalum, surgical steel). The β-radiation emitted has only a short range in the tissue (a few millimeters). In contrast to γ-radiation, it is absorbed very strongly by the tissue, and is therefore very effective. That characteristic of β-radiation makes it possible to keep the total radiation load on the patient very low (applied activity <10 μCi; allowed annual oral intake of ³²P: 600 μCi; integrated radiation dose from stents about 700 Gray) and also makes it possible to confine the radiation, and the treated region, locally. The protective measures required for the treating physician and for transportation, etc., are relatively minor. However, the ion implantation method for stents is technically demanding and cost-intensive. Furthermore, this treatment alone does not solve the problem of thrombus development.

The polymeric compound poly[bis(trifluoroethoxy)phosphazene] exhibits good antithrombogenic action as a filler (see Tur, Untersuchungen zur Thrombenresistenz von Poly[bis(trifluoroethoxy)phosphazenen] [Studies of resistance of poly[bis(trifluoroethoxy)phosphazene] to thrombus formation] and Hollemann Wiberg, “Stickstoffverbindungen des Phosphors” [Nitrogen compounds of phosphorus], Lehrbuch der anorganischen Chemie [Textbook of Inorganic Chemistry], 666-669, 91st-100th Edition, Walter de Gruyter Verlag, 1985; and Tur, Vinogradova et al., “Entwicklungstendenzen bei polymeranalogen Umsetzungen von Polyphosphazenen” [Trends in development of polymer-like reactions of polyphosphazenes], Acta Polymerica 39, No. 8, 424-429 (1988)). Polyphosphazenes are also used in German Patent 196 13 048 for coating artificial implants, without the possibility of making this material therapeutically active by appropriate alteration. Also, this substance alone cannot limit or reduce cell growth leading to restenoses. Furthermore, this polymeric compound, as a purely filler material, does not have the hardness and mechanical strength required, for instance, for artificial heart valves or for stents. But it can be used, in combination with the therapeutic action of isotopic radiation, in other implants or therapeutic devices or means directed toward preventing excessive cellular proliferation.

Therefore this invention is based on the objective of providing a material for medical devices such as catheters, emplastrums, implants, and the like, and for coating them, which should, on one hand, have outstanding mechanical characteristics and antithrombogenic properties so as to improve the biocompatibility of such devices; and, on the other hand, should also prevent or reduce the previously mentioned sequelae of successful treatment or implantation. In particular, uncontrolled cell growth leading, for example, to restenoses following stent implantation, should be prevented or reduced.

This objective is attained by provision of an antithrombogenic polymer with the following general formula (I),

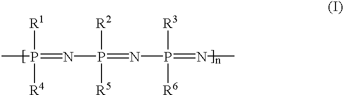

in which

n is from 2 to ∞

R¹ to R⁶ are the same or different and indicate an alkoxy, alkylsulfonyl, dialkylamino or aryloxy group, or a heterocycloalkyl or heteroaryl group in which nitrogen is the heteroatom,

and in which at least part of the polymer chain of the antithrombogenic polymer contains a radiolabeled component.

It is preferable for the radiolabeled component in the antithrombogenic polymer to emit β-radiation in its radioactive decay. However, γ-radiation can also be emitted, depending on the isotope used. In one preferred embodiment, the antithrombogenic polymer contains a radioactive isotope of phosphorus. It is even more preferred for the antithrombogenic polymer to be labeled with ³²P. The phosphorus isotope can be randomly distributed within the polyphosphazene backbone. In another embodiment, every phosphorus in the polyphosphazene backbone, i. e., in the polymer chain of the antithrombogenic polymer, is a radioactive phosphorus isotope. In another embodiment, part of the phosphorus in the antithrombogenic polymer can be replaced by a radioactive arsenic isotope, preferably ⁷⁶As, or by a radioactive antimony isotope, preferably ¹²²Sb, in which case the isotope can be randomly distributed over the polymer chain of the antithrombogenic polymer. ³²P is a β-emitter with a maximum energy of 1.7 Mev, a maximum specific activity of 9000 Ci/mmol, and a half-life of 14.29 days. The maximum range of the β-radiation emitted from ³²P is about 8 meters in air. However, the water making up 80-90% of the tissue acts as a shield, attenuating the radiation emitted so that the maximum penetration in the body tissue is not more than a few millimeters. The β-radiation emitted from the phosphorus isotope reduces uncontrolled cell growth which, for example, causes restenosis following stent implantation. This effect can also be attained by use of γ-radiation, such as that from ⁷⁶As or ¹²²Sb.

As noted previously, the degree of polymerization of the polymer according to the invention can be from 2 to ∞. However, the preferred range for the degree of polymerization is from 20 to 150,000, and more preferably, 40 to 70,000.

Preferably at least one of the groups R¹ to R⁶ in the antithrombogenic polymer is an alkoxy group substituted with at least one fluorine atom.

The alkyl group in the alkoxy, alkylsulfonyl and dialkylamino groups are, for example, straight or branched alkyl groups with 1 to 20 carbon atoms, in which the alkyl group can, for example, be substituted with at least one halogen atom, such as a fluorine atom.

Examples of alkoxy groups are the methoxy, ethoxy, propoxy and butoxy groups, which can preferably be substituted with at least one fluorine atom. The 2,2,2-trifluoroethoxy group is particularly preferred. Examples of alkylsulfonyl groups are methyl, ethyl, propyl and butylsulfonyl groups. Examples of dialkylamino groups are dimethyl, diethyl, dipropyl and dibutylamino groups.

The aryl group in the aryloxy group is, for example, a compound with one or more aromatic ring systems, in which the aryl group can, for example, be substituted with at least one alkyl group as previously defined. Examples of aryloxy groups are the phenoxy and naphthoxy groups and their derivatives.

The heterocycloalkyl group is, for instance, a ring system containing 3 to 7 atoms, with at least one ring atom being a nitrogen atom. The heterocycloalkyl group can, for example, be substituted with at least one alkyl group as previously defined. Examples of heterocycloalkyl groups are the piperidinyl, piperazinyl, pyrrolidinyl and morpholinyl groups and their derivatives. The heteroaryl group is, for example, a compound with one or more aromatic ring systems in which at least one ring atom is a nitrogen atom. The heteroaryl group can, for example, be substituted with at least one alkyl group as previously defined. Examples of heteroaryl groups are the pyrrolyl, pyridinyl, pyridinolyl, isoquinolinyl and quinolinyl groups and their derivatives.

In one preferred embodiment of this invention, the antithrombogenic polymer is a poly[bis(trifluoroethoxy)phosphazene] labeled with ³²P or As or Sb isotopes.

A further object of this invention is the use of the antithrombogenic polymers according to the invention with the general formula (I) as components of therapeutic means to prevent excessive cell proliferation or scarring, or for tumor treatment. In particular, the antithrombogenic polymer according to the invention with the general formula (I) can be used as a component of therapeutic devices such as artificial implants, emplastrums, heart valves, artificial blood vessels, stents, catheters, or urethral or other implants without direct blood contact.

The antithrombogenic polymer according to the invention can, however, be used not only as a coating, but even as the complete material in particular applications, such as in their use as endovascular prostheses and the like. Furthermore, this material can be used not only in arteries, but also in veins and, quite generally, for coating of implants of all types.

Furthermore, according to this invention a therapeutic means is provided which comprises the antithrombogenic polymer according to the invention. Examples of such therapeutic means are emplastrums or additives to them used, in particular, for treatment of increased cell proliferation during wound healing (keloids) or to treat various forms of skin cancers, or an artificial implant.

One preferred embodiment of this invention provides an artificial implant material, which comprises an implant material as a substrate and a biocompatible coating containing the radiolabeled antithrombogenic polymer with the previously specified general formula (I) applied on at least part of the surface of the substrate.

The biocompatible coating of the artificial implant according to the invention has, for example, a thickness of about 1 nm up to about 100 μm, preferably up to about 10 μm, and particularly preferably up to about 1 μm.

There is no particular limitation on the implant material used as the substrate according to the invention. It can be any implant material, such as plastics, metals, metal alloys and ceramics. For instance, the implant material for an artificial heart valve can be pyrolyzed carbon, or a metallic stent material.

In one embodiment of the artificial implant according to the invention, there is a layer to promote adhesion between the surface of the substrate and the biocompatible coating containing the radiolabeled polyphosphazene derivative.

The adhesion promoter or spacer is, for example, a silicon-organic compound, preferably an amino-terminated silane or based on aminosilane, or an alkylphosphonic acid. Aminopropyltrimethoxysilane is particularly preferred.

The adhesion promoter particularly improves adhesion of the coating to the surface of the means or implant material by coupling the adhesion promoter to the surface of the implant material, for instance by ionic and/or covalent bonds and by further coupling of the adhesion promoter to reactive components, particularly to the radiolabeled antithrombogenic polymer of the coating, for instance, through ionic and/or covalent bonds.

The artificial implants according to the invention are produced by applying radioactively labeled polydichlorophosphazene to the surface of the substrate and reacting it with at least one reactive compound selected from aliphatic or aromatic alcohols or their salts, alkylsulfones, dialkylamines and aliphatic or aromatic heterocycles with nitrogen as the heteroatom.

The aliphatic alcohols are, for example, straight or branched monofunctional or polyfunctional alcohols with 1 to 20 carbon atoms, which alcohols can, for instance, be substituted with at least one halogen atom such as a fluorine atom. Alcoholates with alkali metals as cations can be used as the salts of the alcohols, for instance. Preferably the applied radiolabeled polydichlorophosphazene is esterified with sodium 2,2,2-trifluoroethoxide as the reactive compound.

The alkyl groups of the alkylsulfones and dialkylamines are, for example, straight or branched alkyl groups with 1 to 20 carbon atoms, which alkyl groups can be substituted with at least one halogen atom such as a fluorine atom.

Examples of alkylsulfones are methyl, ethyl, propyl and butylsulfone. Examples of dialkylamines are dimethyl, diethyl, dipropyl and dibutylamine. The aromatic alcohols are, for instance, compounds with one or more aromatic ring systems, in which the aromatic alcohols can for instance be substituted by at least one alkyl group as defined above. Examples of aromatic alcohols and their salts are phenol or phenolates and naphthols or naphtholates.

The aliphatic heterocycles are, for example, ring systems containing 3 to 7 atoms, with at least one ring atom being a nitrogen atom. The aliphatic heterocycles can, for instance, be substituted with at least one alkyl group as defined above. Examples of aliphatic heterocycles are piperidine, piperazine, pyrrolidine, morpholine, and their derivatives.

The aromatic heterocycles are, for instance, compounds with one or more aromatic ring systems in which at least one ring atom is a nitrogen atom. The aromatic heterocycles can, for instance, be substituted by at least one alkyl group as defined above. Examples of aromatic heterocycles are pyrrole, pyridine, pyridinol, isoquinoline and quinoline and their derivatives.

Preparation of poly[bis(trifluoroethoxy)phosphazene] is known in the state of the art. Polymerization of hexachlorocyclotriphosphazene is described extensively in Korsak, Vinogradova, Tur, Kasarova, Komarova and Gilman, “Über den Einfluss von Wasser auf die Polymerisation von Hexachlorocyclotriphosphazen” [On the effect of water on the polymerization of hexachlorocyclotriphosphazene], Acta Polymerica 30, No. 5, pages 245-248, 1979. Esterification of the polydichlorophosphazene produced by the polymerization is described by Fear, Thower and Veitch in Journal of the Chemical Society, 1958, page 1324.

The radioactively labeled polyphosphazene derivatives used according to the invention can be prepared by condensation of ³²P-labeled phosphorus pentachloride, either as the pure substance or mixed with unlabeled phosphorus pentachloride, with ammonium chloride. Radioisotopes of arsenic pentachloride or antimony pentachloride can also be used in this step. The quantity of radioisotope, a few micrograms of the isotope, depends on the desired activity. It does not affect the mechanical, chemical and antithrombotic properties of the poly phosphazene derivative.

In the next step, the radiolabeled hexachlorocyclotriphosphazene obtained in the preceding step, by the methods described at the previously mentioned state of the art is polymerized. Then the radiolabeled polydichlorophosphazene produced by polymerization is esterified by methods described in the previously mentioned state of the art.

To produce the artificial implants according to the invention, a previously defined adhesion promoter is applied to the surface of the substrate and coupled to the surface by ionic and/or covalent bonds. Then the radiolabeled polydichlorophosphazene is applied to the surface of the substrate coated with the adhesion promoter, which couples to the radiolabeled polydichlorophosphazene through ionic and/or covalent bonds. Then the radiolabeled polydichlorophosphazene is reacted with at least one of the reactive compounds previously defined.

Preferably the radiolabeled polydichlorophosphazene is applied to the surface of the substrate under an inert gas atmosphere to produce the artificial implant according to the invention, optionally coupled to the adhesion promoter and reacted with the reactive compound. Furthermore, the radiolabeled polydichlorophosphazene can be applied and optionally coupled to the adhesion promoter under reduced pressure or in an air atmosphere.

The radiolabeled polydichlorophosphazene can be applied by wet chemistry, or in solution, or from the melt, or by sublimation, or by spraying, and optionally coupled to the adhesion promoter to produce the artificial implant according to the invention.

The adhesion promoter can be applied to the substrate by wet chemistry or in solution or from the melt or by sublimation or by spraying. The wet-chemical coupling of an adhesion promoter, preferably based on aminosilanes, to hydroxlated surfaces is described in Marco Mantar, Thesis, p. 23, University of Heidelberg, 1991. However, other adhesion promoters known from the state of the art, as well as reagents used as spacers, can be used.

The radiolabeled antithrombogenic polymer can also be applied directly to the surface of the substrate to produce the artificial implant according to the invention.

Also, if an adhesion promoter is used, the adhesion promoter can first be applied to the surface of the substrate, as stated above, and then optionally coupled, after which the radiolabeled antithrombogenic polymer can be applied to the surface of the substrate coated with the adhesion promoter and optionally coupled to the adhesion promoter.

It is preferred to apply the antithrombogenic polymer by wet chemistry or in solution or from the melt and optionally to couple it to the adhesion promoter to produce the artificial implant according to the invention.

The surface of the substrate can be cleaned oxidatively prior to applying the radiolabeled polydichlorophosphazene, the adhesion promoter, or the radiolabeled antithrombogenic polymer. Oxidative cleaning of substrates with simultaneous hydroxylation, as can be used, for instance, for plastic, metallic or ceramic implants, is described in Ulman Abraham, Analysis of Surface Properties, “An introduction to ultrathin organic films”, 108, 1991.

In summary, it is found that the radiolabeled implants according to the invention, especially stents, heart valves, artificial blood vessels, or other implants without direct blood contact can be produced simply and advantageously by means of the process described above. The technically demanding ion implantation of radioactive material such as ³²P into the implant material is not required. Instead, for example, the material emitting β-radiation is applied as a polymeric coating. Both the ³²P-polyphosphazenes and the adhesion promoter can be applied by using processes known from the field of coating, such as spin coating, blade coating, etc.

The implants according to the invention, surprisingly, exhibit the outstanding mechanical properties of the substrate material of the means or implant material. Due to the coating containing the antithrombogenic polymer according to the invention, applied, for example, by direct depositon from the solution, the implants according to the invention not only exhibit antithrombogenic properties, which drastically improves the biocompatibility of such artificial implants, but they also reduce uncontrolled cell growth because of the radiation emitted. Such cell growth causes restenoses following stent implantation, for example.

It has also been found that, for example, radiolabeled poly[bis(trifluoroethoxy)phosphazene] can be immobilized directly with or without adhesion promoters by wet chemistry or by fusion. The success of these preparation steps can be demonstrated by X-ray photoelectron spectrometry.

Both direct coating or coating from the melt with, for instance, radiolabeled poly[bis(trifluoroethoxy)phosphazene], as well as the deposition of radiolabeled polydichlorophosphazene and esterification with, for instance, sodium 2,2,2-trifluoroethoxide

can be carried out with or without a drying step in vacuum or in air or protective gas, in the temperature range from about −20° C. to about 300° C., preferably 0° C. to 200° C., and especially preferably from 20° C. to 100° C., and

can be carried out over a wide range of concentration of the starting material and with different time intervals; for example, from the melt or solutions in appropriate solvents for poly[bis(trifluoroethoxy)phosphazene], polydichlorophosphazene and sodium 2,2,2-trifluoroethoxide, preferably from melts of the pure material and from, for instance, 0.01 molar solutions, over a period of from 10 seconds to 100 hours.

This invention is explained in more detail below by means of examples.

For oxidative cleaning and simultaneous hydroxylation of the surfaces of the artificial implants, the substrate is immersed for 2 hours in a 1:3 mixture of 30% H₂O₂ and concentrated sulfuric acid (Caro's acid) at a reaction temperature of 80° C. Following that treatment, the substrate is washed with 0.5 liter of 18 Mohm-cm deionized water at about pH 5 and then dried in a flow of nitrogen. This cleaning and oxidation step is done as the first step in the following examples according to the invention, if not otherwise specified.

The procedures for working with radioactive materials can be found in textbooks on radiochemical procedures. Other information about the necessary and legally prescribed actions, protective measures, and disposal requirements can be found in the German Regulation on Radiation Protection. These measures apply from the moment on which work with radioactive isotopes is begun.

The ³²P-labeled polydichlorophosphazene which is the basis for the radiolabeled poly[bis(trifluoroethoxy)phosphazene] can be prepared by methods described at the state of the art, beginning with condensation of ³²PCl₅, either the isotopically pure substance or mixed with ordinary, i. e., not radiolabeled, PCl₅, with NH₄Cl. The subsequent polymerization of the radiolabeled hexachlorocyclotriphosphazene is done in an ampule 5 mm in diameter at 250° C.±1° C. with a pressure of 10⁻² mm Hg in the ampule.

### EXAMPLE 1

A 0.1 M solution of ³²P-labeled polydichlorophosphazene is prepared under an inert gas (0.174 g in 5 ml solvent). Absolute toluene is used as the solvent. Then the oxidatively cleaned artificial implant is placed into this solution, under inert gas, at room temperature, for 24 hours. Then the radiolabeled polydichlorophosphazene immobilized on the artificial implant in that manner is esterified with sodium 2,2,2-trifluoroethoxide in absolute tetrahydrofuran as the solvent (8 ml absolute tetrahydrofuran, 0.23 g sodium, 1.46 ml 2,2,2-trifluoroethanol). The reaction mixture is boiled under reflux for the entire reaction period. The esterification is carried out under inert gas at 80° C. over a reaction time of 3 hours. Then the substrate, coated in that manner, is washed with 4-5 ml absolute tetrahydrofuran and dried in a stream of nitrogen.

After these treatments, the surface was examined for its elemental composition, stoichiometry and thickness using X-ray photoelectron spectrometry. The results show that all the reaction steps were completed and coating thicknesses greater than 3.4 nm were attained.

### Example 2

The artificial implant, oxidatively cleaned with Caro's acid, is immersed for 30 minutes in a 2% solution of aminopropyltrimethoxysilane in absolute ethanol. Then the substrate is washed with 4-5 ml absolute ethanol and left in a drying oven for 1 hour at 105° C.

After the coupling of the aminopropyltrimethoxysilane to the oxidatively cleaned surface of the substrate, the treated substrate is placed in a 0.1 M solution of radiolabeled polydichlorophosphazene in absolute toluene for 24 hours at room temperature, under inert gas. Then the treated artificial implant is washed under inert gas with 4-5 ml absolute toluene. Next it is placed in a freshly prepared solution of sodium 2,2,2-trifluoroethoxide (8 ml absolute tetrahydrofuran, 0.23 g sodium, and 1.46 ml 2,2,2-trifluoroethanol) and refluxed at 80° C. for 3 hours in inert gas. Finally, the artificial implant prepared in this manner is washed with 4-5 ml absolute tetrahydrofuran and dried in a stream of nitrogen.

After this treatment, the surface was examined by photoelectron spectrometry for its elemental composition, stoichiometry and coating thickness. The results show that the couplings were accomplished and that coating thicknesses greater than 5.5 run were attained.

### Example 3

The artificial implant, oxidatively cleaned with Caro's acid, is immersed for 30 minutes at room temperature in a 2% solution of aminopropyltrimethoxysilane in absolute ethanol. Then the substrate is washed with 4-5 ml absolute ethanol and left in a drying oven for one hour at 105° C. After coupling of the aminopropyltrimethoxysilane to the surface of the substrate, the artificial implant thus treated is placed for 24 hours at room temperature in a 0.1 M solution of radiolabeled poly[bis(trifluoroethoxy)phosphazene] in ethyl acetate (0.121 g in 5 ml ethyl acetate). Then the artificial implant thus prepared is washed with 4-5 ml ethyl acetate and dried in a stream of nitrogen.

After this treatment, the surface was examined by photoelectron spectrometry for its elemental composition, stoichiometry and coating thickness. The results show that the radiolabeled poly[bis(trifluoroethoxy)phosphazene] was immobilized on the aminopropyltrimethoxysilane adhesion promoter, and that coating thicknesses greater than 2.4 nm were attained.

### Example 4

The artificial implant oxidatively cleaned with Caro's acid is placed for 24 hours in a 0.1 M solution of radiolabeled poly[bis(trifluoroethoxy)phosphazene] in ethyl acetate (0.121 g in 5 ml ethyl acetate) at 70° C. Then the artificial implant so treated is washed with 4-5 ml ethyl acetate and dried in a stream of nitrogen.

The artificial implant so prepared was examined for its elemental composition, stoichiometry and coating thickness using photoelectron spectrometry. The results show that the coupling of the radiolabeled poly[bis(trifluoroethoxy)]phosphazene] to the implant surface was successful and that coating thicknesses greater than 2.1 nm were attained.

### Example 5

The artificial implant oxidatively cleaned with Caro's acid is placed into a melt of the radiolabeled poly[bis(trifluoroethoxy)phosphazene] and left for from about 10 seconds to about 10 hours. Then the implant so treated is washed with 4-5 ml ethyl acetate and dried in a stream of nitrogen.

The artificial implant so prepared was examined for its elemental composition, stoichiometry and coating thickness using photoelectron spectrometry. The results show that the coupling of the radiolabeled poly[bis(trifluoroethoxy)]phosphazene] to the implant surface was successful and any desired coating thicknesses up to a few millimeters were attained.

We claim:

1. Antithrombogenic polymer with the following general formula (I) 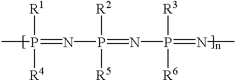
in which
n stands for 2 to ∞,
R¹ to R⁶ are the same or different and mean an alkoxy, alkylsulfonyl, dialkylamino or aryloxy group or a heterocycloalkyl or heteroaryl group with nitrogen as the heteroatom, and
in which at least part of the polymer chain of the antithrombogenic polymer has a radioactively labeled component.

2. Antithrombogenic polymer according to claim 1, in which the radiolabeled component emits β-radiation or γ-radiation on its radioactive decay.

3. Antithrombogenic polymer according to claim 1, which contains a radioactive isotope of the 5th principal group.

4. Antithrombogenic polymer according to claim 3, containing a radioactive phosphorus isotope.

5. Antithrombogenic polymer according to claim 4, in which the phosphorus isotope is ³²P

6. Antithrombogenic polymer according to claim 5, in which the ³²P phosphorus isotope is randomly distributed through the polymer chain.

7. Antithrombogenic polymer according to claim 5, in which every phosphorus atom in the polymer chain of the antithrombogenic polymer is a ³²P isotope.

8. Antithrombogenic polymer according to claim 1, in which at least one of the groups R¹ to R⁶ is an alkoxy group substituted with at least one fluorine atom.

9. Antithrombogenic polymer according to claim 1, which is ³²P-labeled poly[bis(trifluoroethoxy)phosphazene].

10. Use of the antithrombogenic polymer according to claim 1 as a component of therapeutic means to prevent excessive cell proliferation or scarring, or for tumor treatment.

11. Use according to claim 10, in which the means is selected from artificial implants, emplastrums, heart valves, artificial blood vessels, stents, catheters, or ureters or other implants without direct blood contact.

12. Therapeutic means comprising an antithrombogenic polymer according to claim 1.

13. Means according to claim 12 which is an emplastrum.

14. Means according to claim 12 which is an artificial implant.

15. Means according to claim 14 in which the artificial implant comprises an implant material as the substrate and a biocompatible coating containing the above-defined antithrombogenic polymer applied to at least part of the surface of the substrate.

16. Means according to claim 15 in which a layer containing an adhesion promoter is placed between the surface of the substrate and the biocompatible coating.

17. Means according to claim 16 in which the adhesion promoter is a silicon-organic compound.

18. Means according to claim 17 in which the silicon-organic compound is aminopropyltrimethoxysilane.
